SEQUENCE LISTING (32 sequences)

SEQ ID NO: 1 - DNA, 1203 bp, Pseudomonas sp. 101 WT
  source 1..1203: mol_type = genomic DNA
atggctaaagttttatgtgtattatatgatgacccagtagatggttatcctaaaacatatgctcgagatgacttacctaaaattgatcattatcctggtggacaaactctaccaactcctaaagctattgatttcactcctggacaattacttggtagtgtttctggagaattaggtttacgtaaatatcttgaatcaaacgggcatacacttgtagttactagtgataaagatggcccagatagtgtttttgaacgtgaattagtagatgctgatgtagttattagtcaacctttttggccagcttatttaacaccagaacgtattgctaaagcaaaaaacttaaaattagcattaactgcagggatcggaagtgaccatgttgatttacaaagtgcaattgatcgtaatgttactgtagctgaagttacttattgtaattctatttcagtagctgaacatgttgtaatgatgatcttatcattagttcgtaattatctaccttcacacgaatgggcaagaaaaggtggctggaacattgcagactgtgtttcacatgcatatgatctagaagcaatgcatgttggcacagttgctgcaggacgtattggattagctgttttacgtcgattagcaccatttgacgtacatttacactatacagatagacaccgtttaccagagtcagttgaaaaagaattaaatttaacatggcatgctactcgtgaagatatgtatcctgtatgtgatgtagttactttaaattgtccattacatcctgaaacagaacatatgataaatgatgaaactttaaaattattcaaacgaggtgcttatattgtaaatacagctcgtggtaaattatgtgatcgagatgcagttgcaagagctttagaaagtggtcgtctagcaggatatgctggtgatgtttggttccctcaaccagcacctaaagatcacccatggcgtactatgccatataatggtatgacaccacatatttcaggtactacattaactgctcaagctcgatatgctgcaggaactcgtgaaattttagaatgttttttcgaaggtcgtccaattcgagatgaatatttaattgttcaaggtggggcgttagcaggaactggtgcacattcttatagtaaaggcaatgcaacaggtggctctgaagaggctgcaaaatttaaaaaagcagta

SEQ ID NO: 2 - protein, 401 aa, Pseudomonas sp. 101 WT
MAKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 3 - DNA, 1203 bp, Pseudomonas sp. 101
  source 1..1203: mol_type = other DNA
atggctaaagtactttgtgttttatatgatgacccagttgacggttatccaaaaacttacgctcgtgatgaccttcctaaaattgatcattacccaggagggcaaactttaccaacaccaaaagctattgatttcacacctggtcaattacttggttcagtttcaggtgaattaggcttacgaaaatatttagaatctaacggtcatacattagttgtaacttctgataaagatggtccagatagtgtatttgaacgtgaattagttgatgctgatgtagttatttcacaacctttctggccagcttatttaacaccagaaagaatcgcaaaagctaaaaatttaaaacttgctttaacagctggtattggatctgatcacgttgatttacaaagtgctatagatcgaaatgtaactgttgcagaagttacatactgtaattctatttcagttgctgaacacgttgtaatgatgatattatcattagttcgtaattatttaccatcacatgaatgggcaagaaaaggtggatggaatattgcagattgtgtttcacatgcatatgacttagaagcaatgcatgttggaacagttgctgcagggcgtattggattagctgttttacgtcgattagctccatttgatgtacatttacattatacacaacgtaatcgtttaccagaatcagttgaaaaagaattaaatttaacatggcacgcaactcgtgaagacatgtatccagtatgtgatgtagttacattaaattgtcctcttcatccagagacagaacatatgattaatgatgaaacacttaaattatttaaaagaggtgcatatattgttaatactgctcgtgggaaattatgtgatcgtgatgcagttgcacgagctttagaatctggtcgtttagctggatatgcaggtgatgtttggttcccacaacctgcaccaaaagatcatccatggcgtactatgccatataatggtatgacaccacatatttcaggtacaactttaacagcacaagcacgatacgctgcagggactcgagaaatcttagagtgttttttcgaaggtcgaccaattagagatgaatatctaattgtacaaggtggagcattagctggaacaggtgctcattcttattcaaaaggtaatgctacaggtggctcagaagaggcagctaaatttaaaaaagcagtt

SEQ ID NO: 4 - protein, 401 aa, Pseudomonas sp. 101
MAKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTQRNRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 5 - DNA, 1203 bp, Mycobacterium vaccae
  source 1..1203: mol_type = other DNA
atggcaaaagtattatgtgtattatatgatgacccagttgatggatatccaaaaacttacgctcgtgatgacttaccaaaaattgatcattatccaggcggtcaaattttaccaactccaaaagctattgattttacaccaggtcaattacttggttctgtatctggagaacttgggttacgtgaatacctagaatcaaatggtcatacattagttgtaacttcagataaagatggtccagatagtgtttttgaacgagaattagttgatgcagacgttgtaattagtcaaccattttggccagcatatttaactccagaacgaattgcgaaagcgaaaaatttaaaattagcattaacagcaggtattggatctgatcatgttgatttacaaagtgcaattgatcgtaatgttactgttgctgaagttacatatagtaatagtattagtgtagctgaacatgttgtaatgatgattttaagtttagttcgtaattatctaccaagtcatgaatgggcacgtaaaggaggttggaatattgcggactgtgtaagtcatgcgtatgatttagaagcaatgcatgtaggaactgtagcagctggtcgtattggtttagcagtattacgtcgactagctccattcgatgtacacttacattatacacaacgacatcgtttaccagaatcagttgaaaaagaattaaacttaacatggcatgctactcgtgaagatatgtatcctgtttgcgatgttgtaacattaaatgttcctttacatccagaaactgaacatatgattaacgatgaaactttaaaattatttaaacgtggtgcttatattgttaatactgcgagaggtaaattatgtgatagagatgcggttgcacgagcattagaaagtggtcgtttagctggttatgctggtgatgtatggtttcctcaaccagctccaaaagatcacccatggcgtacaatgccttataatggtatgactcctcatatttctggcacaactctaactgcacaagcgcgttacgctgcaggtactcgtgaaattttagaatgttttttcgaaggtcgtccaattcgtgatgaatatttaatagttcaaggtggagcgttagcaggtacaggtgcacattcttatagtaaaggtaatgcaacaggtggatcagaagaggcggctaaatttaaaaaagcagta

SEQ ID NO: 6 - protein, 401 aa, Mycobacterium vaccae
MAKVLCVLYDDPVDGYPKTYARDDLPKIDHYPGGQILPTPKAIDFTPGQLLGSVSGELGLREYLESNGHTLVVTSDKDGPDSVFERELVDADVVISQPFWPAYLTPERIAKAKNLKLALTAGIGSDHVDLQSAIDRNVTVAEVTYSNSISVAEHVVMMILSLVRNYLPSHEWARKGGWNIADCVSHAYDLEAMHVGTVAAGRIGLAVLRRLAPFDVHLHYTQRHRLPESVEKELNLTWHATREDMYPVCDVVTLNVPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 7 - DNA, 1200 bp, Paracoccus sp.12A
  source 1..1200: mol_type = genomic DNA
atggcaaaagtagtttgtgttttatatgatgacccagtagacggttatccaacatcatatgctcgtgattctttaccagtaattgaacgatatccagatggtcaaacattaccaacaccaaaagctattgattttgtaccaggatctcttctaggtagtgtaagtggagaattaggattacgtaattatttagaagcacaaggtcatgaattagtagttacatcaagtaaagacggtccagattcagaattagagaaacacttacatgacgcagaagttgtaatttcacaaccattttggcctgcttatttaacagcagaacgaattgcgaaagcaccaaaattaaaattagcattaacagctggtattggtagtgatcacgtagatttacaagctgcgattgatcgtggtattactgtagctgaggttacattctgtaatagtatttcagtaagtgaacatgttgtaatgactgctttaaacttagtacgtaattatacaccatcacacgattgggctgtaaaaggtggatggaatattgctgattgtgtaactcgttcttatgatattgaaggtatgcatgtaggtactgttgcagctggtcgtattggattagcagttttacgtcgatttaaaccattcgggatgcatttacattatactgaccgtcaccgtttaccacgagaggttgaattagagttagatttaacttggcatgaatctcctaaagacatgtttcctgcatgtgatgttgtaacacttaattgtcctttacatcctgaaacagaacatatggttaatgatgaaacattaaaattatttaaacgaggagcttacttagtaaatactgcacgaggaaaattatgtgaccgtgatgcagtagcacgtgcattagaatcaggtcaattagcaggttatggtggcgatgtatggtttccacaaccagcaccacaagatcatccatggcgtactatgccacataatgcgatgacaccacatattagtggcacatcattatcagctcaagcacgttacgcagcgggcacacgtgaaattttagagtgtcattttgaaggtcgtcctattcgtgatgaatatttaattgtacaaggtggctctttagcgggtgtaggtgctcacagttattcaaaaggtaacgcaacaggcggttcagaagaggctgcaaaatttaaaaaagca

SEQ ID NO: 8 - protein, 400 aa, Paracoccus sp.12A
MAKVVCVLYDDPVDGYPTSYARDSLPVIERYPDGQTLPTPKAIDFVPGSLLGSVSGELGLRNYLEAQGHELVVTSSKDGPDSELEKHLHDAEVVISQPFWPAYLTAERIAKAPKLKLALTAGIGSDHVDLQAAIDRGITVAEVTFCNSISVSEHVVMTALNLVRNYTPSHDWAVKGGWNIADCVTRSYDIEGMHVGTVAAGRIGLAVLRRFKPFGMHLHYTDRHRLPREVELELDLTWHESPKDMFPACDVVTLNCPLHPETEHMVNDETLKLFKRGAYLVNTARGKLCDRDAVARALESGQLAGYGGDVWFPQPAPQDHPWRTMPHNAMTPHISGTSLSAQARYAAGTREILECHFEGRPIRDEYLIVQGGSLAGVGAHSYSKGNATGGSEEAAKFKKA

SEQ ID NO: 9 - DNA, 1203 bp, Ancylobacter aquaticus
  source 1..1203: mol_type = genomic DNA
atggctaaagtattatgtgtattatatgatgacccaattgatggctaccctactacatacgctcgtgataatttaccaaaaatagatcattacccaggcgggcaaacattaccaacaccaaaagcaatcgattttacaccaggtacaatgttaggttcagtatctggtgaattaggtttacgtaaatatttagagagtaacggtcatactcttgttgtaacaagtgataaagacggaccagattcagtttttgaaaaagaattagttgatgcagacatagtaatttcacaaccattttggccagcttatttaacacctgagcgttttgctaaagctaaaaatttaaaattagctttaactgcaggtattggctcagaccacgtagacttacaatcagctattgatcgtggtgttactgtagcagaagtaacatattgtaatagtatttctgtagcagaacatgttgtaatgatgatattaggtctagttcgaaattatctaccagcacatgattgggctcgtaaaggcgggtggaacatcgcagattgcgttaaacattcttacgatttagaagctatgagtgttggtacagttgcagctggtcgaattggtttagcagttttaagacgtttagcgccattcgatgtaaaattacattatacagatcgtcaccgtttaccagaatcagttgaaaaagaattaaatttaacatggcacgcatcaccaacagatatgtatccacattgtgacgttgtaacactaaattgtccattacacccagaaacagaacatatggttaatgaagagacattaaaattatttaaacgtggtgcatatattgttaacacagcacgtggaaaattatgtgatcgagatgcaattgcacgtgcattagaaaatggtacattagctggttatgctggtgatgtatggtttcctcaaccagctcctgctgatcatccatggcgtactatggcatggaatggtatgacaccacatatgagtggtacatcacttactgctcaaactagatacgcagcgggtactcgagaaattttagagtgtttctttgaaggtcgtccaattcgtgatgaatatttaattgtacaaggcggtaatttagcgggagttggtgcacattcatatagtaaaggtaacgcaacaggtgggagtgaagaggctggtaaatttaaaaaagcaggt

SEQ ID NO: 10 - protein, 401 aa, Ancylobacter aquaticus
MAKVLCVLYDDPIDGYPTTYARDNLPKIDHYPGGQTLPTPKAIDFTPGTMLGSVSGELGLRKYLESNGHTLVVTSDKDGPDSVFEKELVDADIVISQPFWPAYLTPERFAKAKNLKLALTAGIGSDHVDLQSAIDRGVTVAEVTYCNSISVAEHVVMMILGLVRNYLPAHDWARKGGWNIADCVKHSYDLEAMSVGTVAAGRIGLAVLRRLAPFDVKLHYTDRHRLPESVEKELNLTWHASPTDMYPHCDVVTLNCPLHPETEHMVNEETLKLFKRGAYIVNTARGKLCDRDAIARALENGTLAGYAGDVWFPQPAPADHPWRTMAWNGMTPHMSGTSLTAQTRYAAGTREILECFFEGRPIRDEYLIVQGGNLAGVGAHSYSKGNATGGSEEAGKFKKAG

SEQ ID NO: 11 - DNA, 1203 bp, Thiobacillus sp.
  source 1..1203: mol_type = genomic DNA
atggctaaaattttatgtgtattatatgatgacccagttgatggttatcctaaaacatatgcacgtgatgacttaccaaaaattgatcattatccaggtggacaaactttaccaacaccaaaagcaatagattttacaccaggtcaattactaggatcagtttctggtgagttaggtttacgaaaatatttagaagcaaacggtcatacatttgttgtaacttcagataaagatggtccagatagtgtatttgaaaaagaattagttgacgcagatgtagttatttctcaacctttttggccagcttatttaactccagaacgtatcgctaaagcgaaaaatttaaaattagctttaacagcaggtattggttctgatcatgtagatttacaatctgcaattgatcgtggtatcactgttgcggaagttacttattgtaattcaatttcagttgcagaacacgtagttatgatgattttaggattagttcgaaattatattcctagtcatgattgggcacgtaaaggtggctggaacattgcagattgtgttgaacattcttatgatctagaaggtatgactgttggtagtgttgcagctggtcgtattggtttagcagtattacgtcgattagcaccattcgatgtaaaattacattatactgatcgacatcgtttacctgaagctgttgaaaaagaattagggttagtatggcatgatactcgtgaagatatgtatccacattgcgatgtagttacattaaatgtaccattacatccagaaacagaacatatgattaacgatgaaacacttaaattatttaaacgaggtgcttatattgtaaatacagcgcgaggtaaattagcggatcgtgatgctattgtacgtgctattgaatcaggtcaattagcaggttatgcaggtgacgtttggtttccacaaccagcacctaaagatcatccttggcgtacaatgaaatgggaaggaatgacaccacatatttcaggtacatcattatcagcacaagctagatatgctgcaggtacacgtgaaattttagaatgttttttcgaaggtcgtccaattcgtgatgaatatttaattgtacaaggaggtgcattagcaggtactggtgctcattcttatagtaaaggtaatgcaacaggtggatcagaagaggctgcgaaatttaaaaaagctggt

SEQ ID NO: 12 - protein, 401 aa, Thiobacillus sp.
MAKILCVLYDDPVDGYPKTYARDDLPKIDHYPGGQTLPTPKAIDFTPGQLLGSVSGELGLRKYLEANGHTFVVTSDKDGPDSVFEKELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 13 - DNA, 1092 bp, Candida boidinii
  source 1..1092: mol_type = other DNA
atgaaaattgtattagttttatatgatgcaggaaaacatgctgcagatgaagagaaactatacggttcaacagaaaataaattagggattgcaaattggttaaaagaccaaggtcatgaattaattacaactagtgataaagaaggtgaaacatcagaattagataaacacattcctgatgcagatattataattactacaccatttcatccagcgtatattacaaaagaacgacttgataaagcaaaaaatttaaaattagtagttgtagctggcgttggctctgatcatatagatctagactatattaatcaaactggtaaaaaaataagtgttttagaagtaacaggttcaaatgtagtttcagttgcagaacatgttgtaatgacaatgttagtattagttcgtaacttcgtaccagcacacgaacaaataattaatcacgattgggaagttgcagctattgctaaagatgcatatgatattgagggtaaaactattgcaacaattggagcaggacgaattggctaccgagtattagagcgattactacctttcaatccaaaagaattactttattacgattatcaagctttaccaaaagaagcagaagagaaagtaggtgcgcgtagagttgaaaatattgaagagttagtagctcaagcagatatagttacagttaatgcacctttacacgcgggtacaaaaggtttaataaataaagaattactatcaaaattcaaaaaaggtgcttggctagtaaatacagctagaggtgctatctgtgtagctgaagatgttgcagcggctttagaatcaggtcaactacgaggatatggtggagatgtttggttcccacaacctgctccaaaagatcatccatggcgtgatatgcgtaataaatacggtgcaggaaatgcaatgacaccacattattcaggtacaactttagatgcacaaacacgttatgcagaaggtacaaaaaatattttagaaagtttctttactggtaaatttgattatcgtcctcaagatattatattacttaatggtgaatatgtaacaaaagcatatggtaaacatgataaaaaa

SEQ ID NO: 14 - protein, 364 aa, Candida boidinii
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 15 - DNA, 1092 bp, Candida boidinii
  source 1..1092: mol_type = other DNA
atgaaaattgtattagttttatatgatgcaggaaaacatgcggcagatgaagagaaactatatggatcaacagaaaataaattaggtattgcaaattggttaaaagatcaaggtcatgaattaattacaactagtgataaagaaggtgaaacttcagaattagataaacatattccagatgctgatattatcattacaactccattccatccagcatatattactaaagaaagattagataaagcaaaaaatttaaaattagtagttgtagcaggtgtaggttcagatcatattgatttagattacattaaccaaactgggaaaaaaatttctgtattagaagtaacaggatcaaatgtagtttcagttgctgaacatgttgtaatgactatgttagttttagtacgtaattttgttcctgcacatgaacaaattataaatcatgattgggaagtagctgcgatcgcaaaagatgcttatgatattgaaggtaaaacaatcgctacaattggtgcgggtagaattggttatcgtgttctagaacgactattaccttttaacccaaaagaattactatattacgattatcaagctttaccaaaagaagcagaagagaaagttggggctcgtagagtagaaaatattgaggaacttgttgcgcaagcagatattgttacagttaatgcacctttacatgcaggcacaaaaggtttaatcaacaaagaacttttatcaaaatttaaaaaaggtgcttggttagtaaacacagctcgaggagctattgctgtagcagaagatgttgcagcggcattagaaagtggacaattaagaggttatggaggtgatgtatggtttccacaacctgctccaaaagatcatccatggcgtgatatgcgtaataaatacggcgcaggcaatgctatgacaccacattatagtggtacaactttagacgctcaaactcgttatgctgaaggtacaaaaaatattttagagtcatttttcactggtaaatttgattatcgtccacaagatattatcttacttaacggtgaatacgttacaaaagcatatggtaaacatgataaaaaa

SEQ ID NO: 16 - protein, 364 aa, Candida boidinii
MKIVLVLYDAGKHAADEEKLYGSTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAIAVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 17 - DNA, 1128 bp, Saccharomyces cerevisiae
  source 1..1128: mol_type = genomic DNA
atgagtaaaggaaaagtattacttgttttatatgaaggtggaaaacatgcggaagagcaagaaaaattacttgggtgtattgaaaatgaattaggtattcgtaattttattgaagagcaaggttatgaattagttacaactattgataaagatccagaaccaacatcaacagtagatcgtgaattaaaagatgcagaaattgtaattactacaccattctttccagcatatatttcacgtaatcgtattgcagaagctcctaacttaaaactatgtgttacagctggtgtaggaagtgatcatgtagatcttgaagctgcaaatgaacgtaaaataacagttacagaagtaactggctctaatgtagtttctgtagctgaacacgtaatggctactatcttagtattaattcgtaattataatggaggtcatcaacaagctattaatggagaatgggacattgcaggtgttgctaaaaatgaatatgatttagaagataaaattatatcaacagtaggtgctggtcgtattggttatcgtgttttagaacgtcttgtagcttttaatccaaaaaaattactttattacgattatcaagaattaccagctgaagcaattaaccgattaaatgaagcatctaaattattcaatggtcgtggtgatattgtacaacgtgtagaaaaattagaagatatggttgctcaaagtgatgttgtaactattaattgtccactacataaagattctcgtggtctatttaacaaaaaattaattagtcatatgaaagatggtgcttatttagttaatactgcgagaggagcaatttgtgtagcagaagatgttgctgaagcagtaaaatcagggaaattagcaggttatggtggcgatgtttgggataaacaaccagctccaaaagatcatccatggagaacaatggataataaagaccatgttggaaatgcaatgacagttcatatttcaggtacatcattagatgctcaaaaaagatatgctcaaggtgttaaaaatatattaaatagttatttctctaaaaaatttgattatcgaccacaagatattatagttcaaaatggttcatatgctactcgtgcatatgggcaaaaaaaa

SEQ ID NO: 18 - protein, 376 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 19 - DNA, 1128 bp, Saccharomyces cerevisiae
  source 1..1128: mol_type = other DNA
atgagtaaaggaaaagttttacttgtattatatgaagggggtaaacatgcagaagagcaagaaaaattactaggttgtattgaaaatgagttaggaattcgtaattttattgaagagcaaggttacgaactagttacaactattgacaaagatccagaaccaacatcaactgttgatcgtgaattaaaagatgcagaaattgtaattactacaccttttttcccagcatatattagtcgtaatcgtattgctgaagcaccaaatttaaaactatgtgttacagcgggtgtaggttctgatcacgtagatttagaagcagctaatgaacgtaaaattacagtaactgaagttacaggttcaaacgttgtaagtgttgctgaacacgtaatggcaacaatattagtattaattcgtaattataatggtggacatcaacaagctattaacggtgaatgggacatcgctggtgtagcaaaaaatgaatatgatttagaagataaaattatcagtacagtaggtgcaggtagaattggttatcgtgttttagaacgtcttgttgcatttaatcctaaaaaattactttattacgcgcgtcaagaattacctgcagaagctattaatcgtttaaatgaagcatctaaattattcaatgggagaggagatattgtacaacgtgttgaaaaattagaagacatggttgctcaatctgatgttgtaactattaattgtccattacataaagattcacgtggtttattcaataaaaaattaatttcacatatgaaagacggtgcatatttagtaaacacagctcgtggagctatttgtgtagctgaagatgtagcagaagctgttaaatctggtaaattagcaggctatggtggggacgtatgggataaacaaccagcaccaaaagatcatccatggagaacaatggataataaagatcatgttggaaatgctatgactgtacacatatcaggtacatcattagatgcgcaaaaacgatatgcacaaggcgtaaaaaatattttaaattcatatttcagtaaaaaattcgattatcgacctcaagatataattgttcaaaatggtagttatgcaactcgtgcatatggtcaaaaaaaa

SEQ ID NO: 20 - protein, 376 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYARQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 21 - DNA, 1206 bp, Moraxella sp. C1 -reco
  source 1..1206: mol_type = genomic DNA
atggcgaaagtagtttgtgttctttacgatgacccaattaacggctatccaacatcatacgcacgtgatgacttacctagaattgataaatatccagatggacaaactttacctacacctaaagcaattgattttactccaggtgctttactaggtagtgtttcaggagaattagggttacgtaaatatcttgaatctcaaggtcatgaattagttgtaacttcttcaaaagatggtcctgattcagaactagaaaaacatttacatgatgcagaagtaatcatttcacaaccattttggccagcttatttaacagcagaaagaatcgcaaaagctccaaaattaaaattagcattaactgctggtattggttcagatcatgtagatcttcaagctgcaattgataacaatattactgtagctgaagttacttattgtaatagtaattcagttgctgaacatgtagttatgatggttttaggtttagtacgtaattacattccatcacatgactgggctcgtaatggtggctggaacattgctgattgtgtagcacgttcttatgatgttgaaggtatgcatgttggtacagttgctgcgggtcgtataggtttacgtgtattacgtcttttagcaccatttgatatgcacttacactatactgaccgtcatcgtttaccagaagctgttgaaaaagaattaaatttaacatggcatgcaacacgtgaggacatgtatggtgcttgtgatgtagttacattaaattgtcctttacatcctgagacagaacatatgattaacgatgaaacacttaaattatttaaacgtggtgcatacttagtaaatacagctagaggtaaattatgtgatcgtgatgcaattgttcgtgctttagaaagtggtagattagcaggctatgcaggtgatgtttggttcccacaaccagcaccaaatgaccatccatggcgtacaatgcctcacaatggtatgacaccacatatttctggaactagtttatcagcacaaacaagatatgctgcgggtacacgtgaaattttagaatgttattttgaaggacgtccaattcgtgatgaatatctaatcgtacaaggaggtggattagctggtgtaggtgcacatagttattcaaaaggtaatgctacaggtggatcagaagaggctgcaaaatatgaaaaattagatgca

SEQ ID NO: 22 - protein, 402 aa, Moraxella sp. C1 -reco
MAKVVCVLYDDPINGYPTSYARDDLPRIDKYPDGQTLPTPKAIDFTPGALLGSVSGELGLRKYLESQGHELVVTSSKDGPDSELEKHLHDAEVIISQPFWPAYLTAERIAKAPKLKLALTAGIGSDHVDLQAAIDNNITVAEVTYCNSNSVAEHVVMMVLGLVRNYIPSHDWARNGGWNIADCVARSYDVEGMHVGTVAAGRIGLRVLRLLAPFDMHLHYTDRHRLPEAVEKELNLTWHATREDMYGACDVVTLNCPLHPETEHMINDETLKLFKRGAYLVNTARGKLCDRDAIVRALESGRLAGYAGDVWFPQPAPNDHPWRTMPHNGMTPHISGTSLSAQTRYAAGTREILECYFEGRPIRDEYLIVQGGGLAGVGAHSYSKGNATGGSEEAAKYEKLDA

SEQ ID NO: 23 - DNA, 1152 bp, Arabidopsis thaliana
  source 1..1152: mol_type = genomic DNA
atggcaatgcgtcaagctgcaaaagctacaattcgtgcttgttcaagttcaagttcttcaggttacttcgcaagacgtcaatttaatgcttcatctggtgattcaaaaaaaattgttggtgttttttataaagctaatgaatatgctacaaaaaatccaaatttcttaggctgtgttgaaaatgctttaggtattagagattggttagaatcacaaggtcaccaatatattgttacagatgacaaagaaggcccagattgtgaattagaaaaacatattcctgacttacatgtattaatcagtactccatttcatccagcttatgtaactgcagaacgtattaaaaaagcaaaaaatttaaaattacttttaacagcgggtattggtagtgatcatattgatttacaagcagctgcagcggcaggtttaactgtagcagaagttactggttcaaatgtagttagtgtagctgaagatgaattaatgcgtattttaattttaatgcgtaattttgtaccaggttataatcaagttgtaaaaggtgaatggaatgttgctgggattgcatacagagcttatgatttagaaggtaaaacaattggcacagttggtgctggtcgtattggtaaattacttttacaacgtttaaaaccattcggatgtaatttactatatcatgatcgtttacaaatggcaccagaattagaaaaagaaacaggagcaaaatttgttgaagatttaaatgaaatgctaccaaaatgtgatgttatagtaattaatatgcctttaacagaaaaaacacgtggaatgttcaataaagaattaattggtaaattaaaaaaaggtgtacttattgttaataacgctcgtggagctattatggaacgacaagcagttgtagatgctgttgaaagtggtcatattggaggttattctggtgatgtttgggatccacaaccagctccaaaagatcacccatggcgttatatgccaaatcaagctatgacacctcatacatcaggcacaactattgatgctcaattacgatatgctgcaggtactaaagacatgctagaacgatattttaaaggtgaagactttccaactgaaaattatattgttaaagatggggaattagctccacaatatcgt

SEQ ID NO: 24 - protein, 384 aa, Arabidopsis thaliana
MAMRQAAKATIRACSSSSSSGYFARRQFNASSGDSKKIVGVFYKANEYATKNPNFLGCVENALGIRDWLESQGHQYIVTDDKEGPDCELEKHIPDLHVLISTPFHPAYVTAERIKKAKNLKLLLTAGIGSDHIDLQAAAAAGLTVAEVTGSNVVSVAEDELMRILILMRNFVPGYNQVVKGEWNVAGIAYRAYDLEGKTIGTVGAGRIGKLLLQRLKPFGCNLLYHDRLQMAPELEKETGAKFVEDLNEMLPKCDVIVINMPLTEKTRGMFNKELIGKLKKGVLIVNNARGAIMERQAVVDAVESGHIGGYSGDVWDPQPAPKDHPWRYMPNQAMTPHTSGTTIDAQLRYAAGTKDMLERYFKGEDFPTENYIVKDGELAPQYR

SEQ ID NO: 25 - DNA, 1164 bp, Glycine max
  source 1..1164: mol_type = genomic DNA
atgagtgatcctactttagctcaaccacatttagttgaagtacatactacattagaaacagtagttactacacataatcataatcatcgtcctagtattaatgcaagtggtgagaaaaagaaaattgtaggtgtattttataaaggtaatgaatatgcaaaactaaatcctaatttcgtaggttgtgttgaaggtgcattaggtattcgagaatggttagaaagtcaaggccatcaatatatcgtaactgacgataaagaaggtcctgatagtgaacttgaaaaacatattccagatgctcatgttatcattagtacaccatttcatcctgcatatgttactgcggaaagaattaagaaagcacaaaaattagaattactattaacagcaggtatcggttcagatcacgtagatttaaaagcagctgcagctgcaggtttaactgtagcagaagttacaggttcaaatgtagtttcagttgcagaagatgaattaatgcgtattttaatccttatgcgaaactttttaccaggttatcatcaagcggttaaaggtgaatggaatgttgctggtattgctcatagagcatatgatttagaaggtaaaactgtaggcacagttggagcaggtcgaattggtaaattactattacaacgtctaaaaccttttaactgtaatctattatatttcgatcgtttacgtattgatcctgaattagaaaaagaaattggagcaaaattcgaggaagatttagacgcaatgttaccaaaatgtgatgttattgttattaatacacctcttacagaacaaacacgtggtttattcgataaaaatcgtattgcgaaatgcaagaaaggtgtattaattgtaaacaatgcacgtggtgcaatcgcagatactcaagcaattgctgacgcttgtagttcaggtcatgttgcaggttattcaggtgatgtttggtttcctcaacctgctccaaaagaccatccatggcgttatatgcctaatcatgcaatgacacctcacatttcagggacaactatcgatgcacaattacgttacgcagcgggtgtaaaagatatgcttgatcgtcattttaaaggtgaagattttccagaacaaaactatattgttaaagaaggacaattagcttctcaatataga

SEQ ID NO: 26 - protein, 388 aa, Glycine max
MSDPTLAQPHLVEVHTTLETVVTTHNHNHRPSINASGEKKKIVGVFYKGNEYAKLNPNFVGCVEGALGIREWLESQGHQYIVTDDKEGPDSELEKHIPDAHVIISTPFHPAYVTAERIKKAQKLELLLTAGIGSDHVDLKAAAAAGLTVAEVTGSNVVSVAEDELMRILILMRNFLPGYHQAVKGEWNVAGIAHRAYDLEGKTVGTVGAGRIGKLLLQRLKPFNCNLLYFDRLRIDPELEKEIGAKFEEDLDAMLPKCDVIVINTPLTEQTRGLFDKNRIAKCKKGVLIVNNARGAIADTQAIADACSSGHVAGYSGDVWFPQPAPKDHPWRYMPNHAMTPHISGTTIDAQLRYAAGVKDMLDRHFKGEDFPEQNYIVKEGQLASQYR

SEQ ID NO: 27 - DNA, 1164 bp, Glycine max
  source 1..1164: mol_type = other DNA
atgagtgatccaacattagctcaacctcatcttgttgaagttcatactacacttgaaacagttgtaactacacataatcacaatcaccgtccaagtatcaacgcatctggtgagaaaaagaaaatagttggtgtattctataaaggaaatgaatatgcaaaactaaatccaaattttgtaggatgtgttgaaggtgcactaggtattcgtgaatggcttgagtcacaaggtcatcaatatatagttactgatgacaaagaaggtccagatagtgaattagaaaaacatattccagatgcacatgttataattagtacaccttttcatccagcttatgttactgctgaacgtattaagaaagctcaaaaactagaactattacttactgctggcattggttcagatcatgttgatttaaaagctgcagctgcagctggtttaactgtagctgaagtaactggtagtaatgtagtttctgtagcagaagatgaactaatgcgtattcttattcttatgcgaaactttttaccaggatatcatcaagcagtaaaaggagagtggaatgtagcaggaattgctcatcgagcttatgatttagaaggtaaaacagttggaacagtaggtgcaggtcgaataggtaaattactattacaacgtttaaaaccatttaattgtaatctattatatttcgatcgattacgtattgatccagaattagaaaaagaaattggtgcaaaatttgaagaggatttagatgctatgttaccaaaatgtgatgttattgttattaatactccattaacagaacaaacacgaggcttattcgataaaaacagaattgcaaaatgtaagaaaggtgttttaattgtaaataacgctcgtggtgcaatagcagatactcaagcaattgctgatgcgtgctcaagtggtcatgttgctggttatagtggagatgtatgggatccacaaccagcacctaaagatcacccatggcgttacatgccaaatcatgcaatgacaccacatatttcaggtacaactattgatgcacaattacgatatgcagctggtgtaaaagatatgttagatcgtcatttcaaaggtgaagattttcctgaacaaaattatatcgtaaaagaaggacaattagcaagtcaatatcgt

SEQ ID NO: 28 - protein, 388 aa, Glycine max
MSDPTLAQPHLVEVHTTLETVVTTHNHNHRPSINASGEKKKIVGVFYKGNEYAKLNPNFVGCVEGALGIREWLESQGHQYIVTDDKEGPDSELEKHIPDAHVIISTPFHPAYVTAERIKKAQKLELLLTAGIGSDHVDLKAAAAAGLTVAEVTGSNVVSVAEDELMRILILMRNFLPGYHQAVKGEWNVAGIAHRAYDLEGKTVGTVGAGRIGKLLLQRLKPFNCNLLYFDRLRIDPELEKEIGAKFEEDLDAMLPKCDVIVINTPLTEQTRGLFDKNRIAKCKKGVLIVNNARGAIADTQAIADACSSGHVAGYSGDVWDPQPAPKDHPWRYMPNHAMTPHISGTTIDAQLRYAAGVKDMLDRHFKGEDFPEQNYIVKEGQLASQYR

SEQ ID NO: 29 - DNA, 1206 bp, Pseudomonas sp. 101
  source 1..1206: mol_type = other DNA
atggcaaaagttttatgtgttctttatgacgatccagtagatggatacccaaaaacatatgcacgagacgatctacctaaaattgaccattatccaggcggtcaaactcttccaacaccaaaagcaattgattttactccaggtcaactattaggatcagtttcaggagaattaggacttcgtaaatatcttgaaagtaatggtcacactttagtagttacttcagataaagacggcccagattcagtatttgaacgtgaattagttgacgcggacgtagttatttcacaaccattttggccagcatatttaacaccagaacgtattgcaaaagcaaaaaatcttaaattagcactaacagcgggtataggtagtgatcatgtagatcttcaaagtgcaattgatcgtaatgtaacagttgctgaagtaacatactgtaattctatcagtgtagcagaacacgttgtaatgatgatcttatcattagttcgaaactatttaccatctcatgaatgggctagaaaaggtggatggaatatcgcagattgtgttagtcatgcgtatgacttagaagcaatgcatgttggtacagtaggagctggacgtattggattagcagttttacgacgtttagcaccatttgatgtacatttacactatacagatagacatcgacttcctgagtcagttgaaaaagaattaaatttaacttggcatgctactcgagaagacatgtatccagtatgtgatgtagttactttaaattgtccattacatccagaaacagaacatatgattaatgatgaaacattaaaattatttaaaagaggcgcttatattgttaatacagctcgaggtaaattatgtgatcgagatgcagttgctagagctttagaaagtggccgtttagcagggtatgctggcgatgtttggtttcctcaaccagctccaaaagatcatccatggcgaacaatgccttataatggaatgacaccacacatttcaggcactacattaactgcacaagctcgttatgcagctggaactcgtgaaattttagaatgttttttcgaaggacgtccaattcgtgacgaatatttaatcgtacaaggaggtgctttagcaggaacaggagctcattcatatagtaaaggtaatgctacaggtgggagtgaggaagctgcgaaatttaaaaaagcagtataa

SEQ ID NO: 30 - protein, 401 aa, Pseudomonas sp. 101
MAKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVGAGRIGLAVLRRLAPFDVHLHYTQRHRLPESVEKELNLTWHATREDMYPVCDVVTLNAPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAKVYSKGNATGGSEEAAKFKKAV

SEQ ID NO: 31 - DNA, 1206 bp, Pseudomonas sp. 101
  source 1..1206: mol_type = other DNA
atggcaaaagttttatgtgttctttatgacgatccagtagatggatacccaaaaacatatgcacgagacgatctacctaaaattgaccattatccaggcggtcaaactcttccaacaccaaaagcaattgattttactccaggtcaactattaggatcagtttcaggagaattaggacttcgtaaatatcttgaaagtaatggtcacactttagtagttacttcagataaagacggcccagattcagtatttgaacgtgaattagttgacgcggacgtagttatttcacaaccattttggccagcatatttaacaccagaacgtattgcaaaagcaaaaaatcttaaattagcactaacagcgggtataggtagtgatcatgtagatcttcaaagtgcaattgatcgtaatgtaacagttgctgaagtaacatactgtaattctatcagtgtagcagaacacgttgtaatgatgatcttatcattagttcgaaactatttaccatctcatgaatgggctagaaaaggtggatggaatatcgcagattgtgttagtcatgcgtatgacttagaagcaatgcatgttggtacagtaggagctggacgtattggattagcagttttacgacgtttagcaccatttgatgtacatttacactatacacaaagacatcgacttcctgagtcagttgaaaaagaattaaatttaacttggcatgctactcgagaagacatgtatccagtatgtgatgtagttactttaaatgctccattacatccagaaacagaacatatgattaatgatgaaacattaaaattatttaaaagaggcgcttatattgttaatacagctcgaggtaaattatgtgatcgagatgcagttgctagagctttagaaagtggccgtttagcagggtatgctggcgatgtttggtttcctcaaccagctccaaaagatcatccatggcgaacaatgccttataatggaatgacaccacacatttcaggcactacattaactgcacaagctcgttatgcagctggaactcgtgaaattttagaatgttttttcgaaggacgtccaattcgtgacgaatatttaatcgtacaaggaggtgctttagcaggaacaggagctaaagtttatagtaaaggtaatgctacaggtgggagtgaggaagctgcgaaatttaaaaaagcagtataa

SEQ ID NO: 32 - protein, 388 aa, Pseudomonas sp. 101; identical to SEQ ID NO: 28

(19) United States
(12) Patent Application Publication
(10) Pub. No.: US 2023/0357704 A1
(43) Pub. Date: Nov. 9, 2023

(54) PHOTOBIOCONVERSION OF ELECTROCATALYSIS-DERIVED FORMATE

(71) Applicant: Alliance for Sustainable Energy, LLC, Golden, CO (US)

(72) Inventors: Lukas Royce DAHLIN, Golden, CO (US); Alexander William MEYERS, Denver, CO (US); Michael T. GUARNIERI, Denver, CO (US)

(21) Appl. No.: 18/311,875

(22) Filed: May 3, 2023

Related U.S. Application Data
(60) Provisional application No. 63/338,000, filed on May 3, 2022.

(51) Int. Cl. C12N 1/12; C12P 21/02; C12M 1/42

(52) U.S. Cl. CPC C12N 1/12; C12P 21/02; C12M 35/02

(57) ABSTRACT

Disclosed herein are compositions and methods for biological upgrading of electrocatalysis-derived formate presents a promising approach to sequester CO₂, valorize curtailed electrons, and establish a sustainable formate bioeconomy.

### CROSS-REFERENCE TO RELATED APPLICATIONS

This application claims priority from U.S. Provisional Patent Application No. 63/338,000, filed on 3 May 2022, the contents of which are incorporated herein by reference in their entirety.

### CONTRACTUAL ORIGIN

The United States Government has rights in this invention under Contract No. DE-AC36-08GO28308 between the United States Department of Energy and the Alliance for Sustainable Energy, LLC, the Manager and Operator of the National Renewable Energy Laboratory.

### SEQUENCE LISTING

The instant application contains a Sequence Listing which has been submitted with the filing of this application and is hereby incorporated by reference in its entirety. The XML copy as filed herewith was originally created on 3 May 2023. The XML copy as filed herewith is named NREL_21-124.xml, is 53,583 bytes in size and is submitted with the instant application.

### BACKGROUND

Rising global greenhouse gas emissions and the impacts of resultant climate change necessitate development and deployment of carbon capture and conversion technologies. Amongst the myriad of bio-based conversion approaches under evaluation, a formate bioeconomy has recently been proposed, wherein CO₂-derived formate serves as a substrate for concurrent carbon and energy delivery to microbial systems. To date, this approach has been explored in chemolithotrophic and heterotrophic organisms via native or engineered formatotrophy. However, utilization of this concept in phototrophic organisms has yet to be reported.

### SUMMARY

In an aspect, disclosed herein is a non-naturally occurring phototrophic organism comprising a non-naturally occurring gene encoding for a formate dehydrogenase enzyme wherein the phototrophic organism can grow on formate as a sole carbon source. In an embodiment, the non-naturally occurring gene has a nucleotide sequence that is greater than 70% identical to SEQ ID NO: 29, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17. In an embodiment, the non-naturally occurring gene expresses a formate dehydrogenase enzyme that has an amino acid sequence that is greater than 70% identical to SEQ ID NO: 30, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18. In an embodiment, the non-naturally occurring gene is incorporated into the plastidial genome of the phototrophic organism. In an embodiment, the organism is Picochlorum renovo sp. In an embodiment, the phototrophic organism exhibits increased growth in a medium comprising carbon dioxide and formate when compared to the corresponding naturally occurring phototrophic organism. In an embodiment, the concentration of carbon dioxide in the medium is less than about 0.04 percent. In an embodiment, the concentration of formate in the medium is greater than about 5 percent. In an embodiment, the formate dehydrogenase enzyme uses NAD+ as a cofactor. In an embodiment, the concentration of formate as a sole carbon source is greater than 10 mM. In an embodiment, the concentration of formate as a sole carbon source is greater than 25 mM. In an embodiment, the concentration of formate as a sole carbon source is greater than 70 mM.

In an aspect, disclosed herein is a method for the growth of a non-naturally occurring phototrophic organism comprising using an electrolyzer to produce formate from carbon dioxide and then contacting the non-naturally occurring phototroph with the produced formate. In an embodiment, the concentration of formate as a sole carbon source is greater than 10 mM. In an embodiment, the concentration of formate as a sole carbon source is greater than 25 mM. In an embodiment, the concentration of formate as a sole carbon source is greater than 70 mM. In an embodiment, the non-naturally occurring phototrophic organism is Picochlorum renovo sp. In an embodiment, the non-naturally occurring phototrophic organism comprises a non-naturally occurring gene encoding for a formate dehydrogenase enzyme. In an embodiment, the non-naturally occurring gene has a nucleotide sequence that is greater than 70% identical to SEQ ID NO: 29, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17. In an embodiment, the non-naturally occurring gene expresses a formate dehydrogenase enzyme that has an amino acid sequence that is greater than 70% identical to SEQ ID NO: 30, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18.

Other objects, advantages, and novel features of the present invention will become apparent from the following detailed description of the invention when considered in conjunction with the accompanying drawings.

### DESCRIPTION OF THE DRAWINGS

FIG. 1 depicts an overview of FDH-mediated photoformatotrophy in P. renovo. FDH is transgenically expressed in the P. renovo chloroplast, enabling conversion of formate to a reducing equivalent and CO₂, which can then be assimilated via native metabolism.

FIG. 2 depicts formate toxicity screening in P. renovo. Growth curves of P. renovo with varying sodium formate concentrations, at pH=6.0 in the presence of 2% CO₂. Data represents the average and standard deviation of 3 biological replicates.

FIG. 3 depicts formate dehydrogenase plastid integration in P. renovo. (Left) Genetic construct for expression of FDH in the chloroplast genome. (Right) PCR amplification of the formate dehydrogenase insert, verifying homoplasmy of the chloroplast genome. WT, wild-type P. renovo; NADP-FDH, transformant P. renovo expressing the NADP-FDH variant; NAD-FDH, transformant P. renovo expressing the NAD-FDH variant.

FIGS. 4a and 4b depict growth and formate utilization analyses for WT and FDH-expressing P. renovo supplemented with 25 mM formate. FIG. 4a (Left) Growth curves of wild type, NADP⁺ FDH, and NAD⁺ FDH-expressing P. renovo with 25 mM sodium formate addition at non-growth-limiting (2%) CO₂ conditions at pH=6.0. FIG. 4b (Right) HPLC analysis of culture supernatant for formate utilization. Data represents the average and standard deviation of 3 biological replicates.

FIGS. 5a and 5b depict growth and formate utilization analyses for WT and FDH expressing P. renovo supplemented with 10 mM formate. FIG. 5a (Left) Growth curves of wild type, and NAD FDH-expressing P. renovo with 10 mM sodium formate addition at non-growth-limiting (2%) CO₂ conditions at pH=6.0. FIG. 5b (Right) HPLC analysis of culture supernatant for formate utilization. Data represents the average and standard deviation of 3 biological replicates.

FIGS. 6a and 6b depict Growth and formate utilization analyses for WT and FDH expressing P. renovo supplemented with 5 mM formate and atmospheric CO₂. FIG. 6a (Left) growth curves of wild type with and without 5 mM sodium formate, and NAD⁺FDH-expressing P. renovo with 5 mM sodium formate addition at ambient (0.04%) CO₂ conditions at a pH of 6.0. FIG. 6b (Right) HPLC analysis of culture supernatant for formate utilization. Data represents the average and standard deviation of 3 biological replicates.

FIG. 7 depicts a schematic overview of photobioelectroconversion of CO₂ to renewable propylene glycol. Electroreduction of CO₂ to formate, using curtailed electrons from wind and solar operations, can be coupled to microalgal photoconversion of C1 substrates. Production of propylene glycol will be enabled via metabolic engineering of P. renovo for heterologous expression of (1) methylglyoxal synthase, (2) methylglyoxal reductase, and (3) 1,2-propanediol reductase.

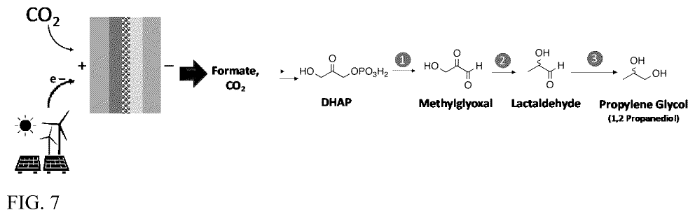

FIGS. 8a and 8b depict growth of FDHs from Pseudomonas sp. 101 WT (SV19), Pseudomonas sp. 101 (D221Q/H223N) (SV20), Mycobacterium vaccae FDH (C145S/D221Q/C225V) (SV21), Paracoccus sp.12A-FDH-WT (SV22), Ancylobacter aquaticus FDH (SV23), Thiobacillus sp. FDH (SV24), Candida boidinii FDH (C23S) (SV26), Candida boidinii FDH (C23S/C262A) (SV27), Saccharomyces cerevisiae FDH-WT (SV28), Saccharomyces cerevisiae FDH (D196A/ Y197R) (SV29), Moraxella sp. C1-reco FDH (SV30), Arabiodopsis thaliana FDH WT (SV31), Gmax (Glycine max) FDH WT (SV32), Gmax (Glycine max) FDH (F290D) (SV33), and Pseudomonas sp. 101 (A198G) (pLRD 176 (NAD FDH)) in different concentrations of formate. FIG. 8a depicts growth of FDHs in 10 mM formate, 2% CO₂ at pH=6 Bis-Tris and 5 mM NH₄Cl. FIG. 8b depicts growth of FDHs in 75 mM formate, 2% CO₂ at pH=6 Bis-Tris and 5 mM NH₄Cl.

### DETAILED DESCRIPTION

As disclosed herein, we have taken the first steps to establish formate utilization in Picochlorum renovo, a recently characterized eukaryotic microalga with facile genetic tools and promising applied biotechnology traits. Plastidial heterologous expression of a formate dehydrogenase (FDH) enabled P. renovo growth on formate as a carbon and energy source. Further, FDH expression enhanced cultivation capacity on ambient CO₂, underscoring the potential for bypass of conventional CO₂ capture and concentration limitations. This work establishes a photoformatotrophic cultivation regime that leverages light energy-driven formate utilization. The resultant photosynthetic formate platform has widespread implications for applied phototrophic cultivation systems and the bio-economy at large.

Development of novel CO₂ sequestration and valorization strategies are urgently needed to reduce greenhouse gas emissions and ameliorate the negative environmental and social impacts of climate change. Indeed, such approaches also present an opportunity to address rapidly increasing global energy and food security demands. To this end, bio-based technologies to convert CO₂ to fuels, chemicals, materials, and food are actively being evaluated. Harnessing the power of microbial metabolism to capture and convert CO₂ represents a high-potential route to enable such bio-based approaches. However, microbial cultivation using CO₂ as a carbon substrate faces a series of challenges, ranging from point source distribution limitations to gas-liquid mass transfer hurdles, and high cellular energy requirements for efficient biological reduction and CO₂ assimilation.

To bypass the hurdles associated with CO₂ bioconversion, the concept of a formate bio-economy has recently been proposed, wherein CO₂-derived formate is converted to the aforementioned commodities by leveraging formatotrophic microbial metabolism. In one envisioned embodiment, a formate bio-economy would entail the use of renewable electricity to capture and electrochemically reduce either atmospheric (via direct air capture) or point source CO₂ emissions to formate. This formate could then be upgraded via a variety of formatotrophic microbes to produce sustainable bioproducts. This approach presents an opportunity to utilize renewable electricity, while sequestering and converting CO₂ to formate, thereby directly reducing greenhouse gas emissions.

To date, formate bioconversion has primarily been evaluated in chemolithotrophic and heterotrophic organisms such as Cupriavidus necator, Escherichia coli, or Saccharomyces cerevisiae, via either native formatotrophy or engineered formatotrophic pathways. For example, microbial formatotrophy has been achieved through FDH-mediated Calvin-Benson-Bassham (CBB) cycle-driven CO₂ fixation that is native in C. necator or engineered into E. coli. Alternatively, higher metabolic efficiency can be achieved via direct formate assimilation pathways (e.g., the reductive glycine pathway). However, these biological systems suffer from reducing power and ATP requirements needed to fix the carbon contained in formate, or incomplete and/or low-yield carbon fixation.

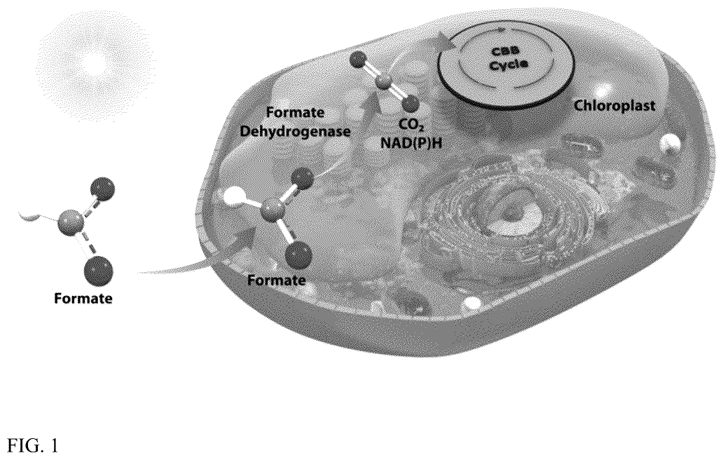

Phototrophic organisms present an intriguing, high-potential route to leverage the power of light energy coupled to formatotrophy to enhance growth. However, to date, photosynthesis-coupled formatotrophy has yet to be established. Herein, we have taken the first steps towards enabling the direct feed of formate as a sole or co-fed carbon and energy source to a phototrophic organism via the integration of a formate dehydrogenase (FDH) into the chloroplast genome of the industrially-relevant microalga, Picochlorum renovo (FIG. 1). The resultant strain is capable of utilizing formate as a carbon and energy source and displays enhanced growth on ambient CO₂ when supplemented with formate.

### Formate Toxicity Screening

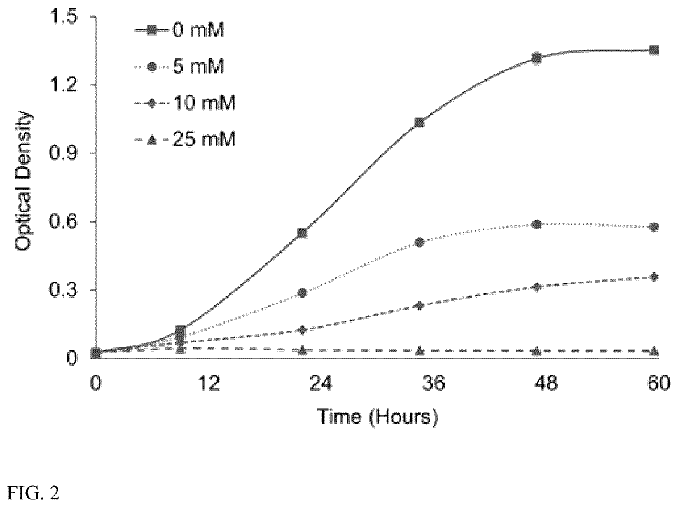

To evaluate formate toxicity and potential for formate utilization in P. renovo, we evaluated growth in the presence of 2% CO₂ with sodium formate supplementation at various concentrations over 60 hours (FIG. 1). Conventionally, P. renovo is cultured at a pH of 7-8 and displays poor growth at pH values <6. However, studies in other organisms have shown that low pH (<7) leads to increased formate transport, either through active transport or enhanced passive diffusion of protonated formic acid. As such, growth was evaluated at pH 6 via Bis-tris buffering. At this pH, concentrations of 5 mM and 10 mM formate reduced P. renovo growth, while a concentration of 25 mM completely inhibited growth (FIG. 2). As previously reported, this toxicity is likely due to formic acid transport into the cell and resultant acidification of the cytoplasm upon dissociation to formate and hydrogen ions.

### Heterologous Formate Dehydrogenase Expression

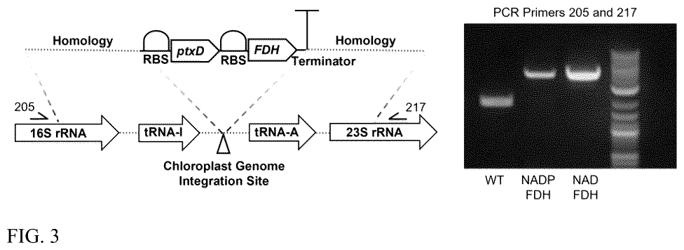

To reduce formate toxicity and enable formate utilization, we sought to establish a mechanism by which formate-derived carbon could be assimilated into P. renovo CBB metabolism via expression of a FDH (FIG. 1). Two FDH mutants previously evolved from the Pseudomonas sp. 101 FDH were identified that preferentially use either NAD+ or NADP+, respectively, as a cofactor for the oxidation of formate to CO₂. These two FDHs were codon optimized to the P. renovo chloroplast genome and assembled into our previously established chloroplast integration vector for constitutive expression utilizing phosphite dehydrogenase (ptxD) as a selectable marker. Transformant algae were obtained via biolistics, and homoplasmy of the chloroplast genomes was confirmed via PCR and Sanger sequencing utilizing primers flanking the insertion site (FIG. 3).

### Formate Utilization Under High CO₂ Cultivation

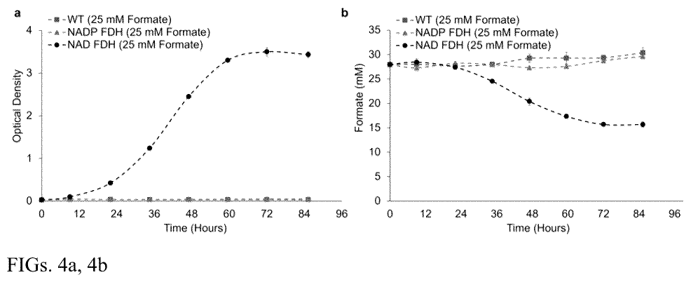

We next evaluated the potential for formate utilization in FDH-expressing strains at non-growth-limiting (2%) CO₂ concentrations (FIG. 4). Growth in media supplemented with 25 mM formate was observed for the NAD+-utilizing FDH variant, with 48±1% of formate consumed from the culture media after 85 hours of cultivation. Conversely, no growth and no formate utilization were observed for the strain expressing the NADP+-utilizing FDH variant. The wild-type culture did not grow on formate and no formate utilization was observed (FIG. 4).

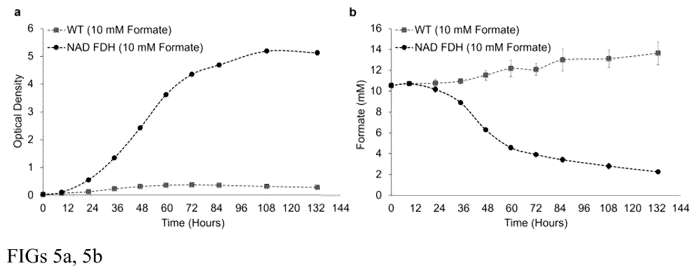

Following down selection to the NAD+-utilizing FDH variant, cultivation capacity on 10 mM sodium formate was assessed to determine if reducing formate levels could decrease residual toxicity and lead to increased growth and percentage of formate utilized. Indeed, a higher culture density was reached when cultivated under 10 mM formate compared to 25 mM formate, potentially due to decreased toxicity when cultivated at lower formate concentrations (FIGS. 4a and 5a). Taking evaporative losses into account, formate consumption of the NAD+FDH strain at 85 hours was 77±2% and 88±1% at 132 hours. Notably, formate utilization was coincident with growth, with most of the formate consumption occurring during the active growth phase of P. renovo (hours 24-72) (FIG. 5).

### Formate Utilization Under Ambient CO₂ Cultivation

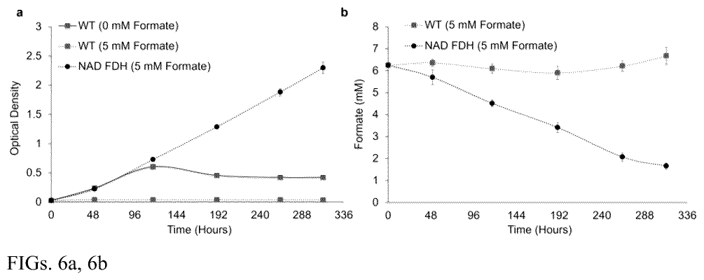

We next analyzed growth at ambient concentrations of CO₂ (0.04%) to determine if exogenously supplemented formate could lead to a growth enhancement under CO₂-limited conditions. P. renovo grows significantly slower when cultivated on air, compared to 2% CO₂, 5 mM formate was thus utilized to compensate for the reduced growth rate (FIGS. 5a and 6a). As shown in FIG. 6, cells expressing FDH displayed enhanced growth when supplemented with 5 mM formate, growing to a higher final culture density than those without formate supplementation. Formate concentrations dropped from an initial starting concentration of 6.2 mM to 1.7 mM after 312 hours, as measured via HPLC. This represents 74+/−2% utilization of the added formate. Under these same conditions, no formate utilization was observed in wild-type cultures (FIG. 6). The initial growth rates of wild-type and NAD+-FDH-expressing strains were equivalent. However, following ˜117 hours of cultivation, the unsupplemented wild-type culture enters stationary phase whereas the supplemented NAD+-FDH-expressing strain continues to grow to >3.8× optical density relative to wild-type.

CO₂ delivery has been predicted to account for nearly 20% of algal biomass production costs and also presents carbon utilization efficiency (CUE) hurdles due to poor gas-liquid mass transfer and rapid off gassing in open systems. Improved carbon delivery and CUE could be achieved via the direct feeding of water-soluble formate to phototrophic systems, which would concurrently deliver necessary carbon and reducing equivalents for growth. Additionally, the relatively low concentration of atmospheric CO₂ can be a key limiting factor in terrestrial phototroph productivity. Therefore, photoformatotrophy could also be deployed in terrestrial crops to enhance productivity in support of a bioeconomy and increasing global food production demands.

To fully bring to bear the potential of photoformatotrophy, a series of key conversion hurdles will require targeted bypass. Enhancement of formate utilization may be achieved by targeting a series of interacting variables, including formate/formic acid transport rate across the cell membrane, which may occur via passive or active transport mechanisms. Additionally, the pool of intracellular oxidizing equivalents in the form of NAD(P)+ can be targeted. Finally, the activity of the expressed FDH may be limiting and presents a high-potential target for protein engineering and screening.

With regard to formate transport, genomic analysis of P. renovo identified a putative formate/nitrite transporter with 39% homology to the fdhC formate transporter in Methanobacterium formicium. This fdhC homolog also encodes a conserved formate/nitrite transporter domain with 6 associated transmembrane domains, which could be responsible for formate transport in this alga, in conjunction with passive diffusion. Genetic engineering and culture optimization for increased formate transport is an area of future work that could be achieved through heterologous expression of various characterized formate transporters, or through manipulation of culture pH to concurrently optimize formate transport and cellular growth. A confounding factor in the work presented here is the fact that as organisms utilize sodium formate, an OH— anion is produced which can increase pH (depending upon the buffering capacity of the culture media), thereby decreasing formate transport as growth occurs. This can be circumvented by the addition of formic acid in pH-stat fed bioreactors, which results in no net change to culture pH as formic acid is utilized.

Alternatively, NAD+ levels may limit FDH activity through a lack of oxidizing equivalents needed for formate oxidation. NAD+ levels in phototrophic systems may be increased through either limiting light intensity or decreasing light absorption by the photosynthetic antenna. However, such approaches could limit photo-productivity. Alternatively, metabolic pathways that require large amounts of reducing equivalents could be upregulated, or novel pathways introduced, such as starch, lipid, or terpenoid biosynthesis, which would in turn produce useful biochemical intermediates while regenerating needed oxidizing equivalents for formate utilization.

Finally, inherent FDH kinetics and cofactor specificity may limit FDH activity, and thus hinder formate utilization. At a high level, known FDH enzymes are separated into two classes, metal-independent, and metal-dependent. While the metal-independent class is generally less cumbersome for heterologous expression, due to single subunit functionality (such as the Pseudomonas variant utilized herein), metal-dependent FDHs are generally more complex and have more favorable kinetics. Localization of the FDH offers a further opportunity for optimization; for example, addition of a RuBisCO binding motif to the FDH may localize the FDH to RuBisCO, such that CO₂ produced from formate oxidation is readily available for fixation by the enzyme. In the results presented herein, the NADP+ utilizing FDH variant did not grow in the presence of formate, suggesting minimal to no functionality. This was unexpected, as NADP+is generally considered to be the most abundant dinucleotide cofactor in the chloroplast. The lack of NADP+ FDH functionality in P. renovo could be due to a higher proportion of NADPH, limiting the availability of non-reduced NADP+ equivalents needed for FDH functionality, or the relatively poor enzyme kinetics of the NADP+-utilizing FDH variant.

In summary, we have taken the first steps towards engineering a phototroph for formatotrophy. This strategy offers the potential for a series of benefits to enhance the productivity of phototrophs via the delivery of reduced carbon in the form of formate that can be readily produced from CO₂ via electrolysis. First, in comparison to gaseous substrates such as CO₂, formate is notably easier to both store and transport. Second, formate is completely miscible in water thereby increasing mass transfer while decreasing potential for CO₂ off gassing which ultimately manifests as low system CUE. Third, formate also enables the ultimate conversion of electrical energy to cellular energy (i.e., reducing equivalents), in turn enabling higher cell density cultivation. Fourth, formate is broadly toxic to many organisms, as such, contamination can be greatly reduced, which can lead to drastic declines in biomass yields during cultivation of both aquatic and terrestrial phototrophs. While a number of these benefits apply to aquatic species, application of formate feeding to higher plants represents an additional exciting area of future work. Finally, this work lays the foundation for incorporation of more efficient, direct formate utilizing pathways, such as the reductive glycine and formolase pathways, and integration with microbial electrosynthesis approaches wherein formate serves as an electron and carbon mediator molecule, to ultimately enable a photosynthetically-driven formate bio-economy.

### Strain and Cultivation Conditions

Formate toxicity screening was carried out utilizing a modification of our previously described media. Media was prepared with 250 mL of seawater (Gulf of Maine, Bigelow Labs), and 750 mL of deionized water. Macro nutrient concentration was 5 mM N (as NH₄C), and 0.313 mM P (as NaH₂PO₄). Trace metals were 1.06×10⁻⁴ M Si (as Na₂SiO₃ 9H₂O)), 1.17×10⁻⁵ M Fe (as FeCl₃ 6H₂O), 1.17×10⁻⁵ M EDTA (as Na₂EDTA 2H₂O), 3.93×10⁻⁸ M Cu (as CuSO₄ 5H₂O)), 2.60×10⁻⁸ M (as Na₂MoO₄ 2H2O), 7.65×10⁻⁸ M Zn (as ZnSO₄ 7H₂O), 4.20×10⁻⁸ M Co (as CoCl₂ 6H2O) and 9.10×10⁻⁷ M Mn (as MnCl₂ 4H₂O). Vitamins were added as follows, thiamine HC1 (2.96×10⁻⁷ M), biotin (2.05×10⁻⁹ M) and cyanocobalamin (3.69×10⁻¹⁰ M). Trace metal, silica and vitamin stock solutions were purchased from Bigelow Labs. Media was buffered with 10 mM Bis-Tris, and media pH was adjusted to 6.0 using concentrated HCl.

Sodium formate (HCO₂Na) was added to the above media to obtain the desired formate concentration. To assay for formate toxicity, 45 mL of culture (in a 250 mL Erlenmeyer flask) was inoculated from mid log phase cells to an optical density (750 nm) of 0.025. Cultures were mixed via shaking (170 rpm) at 33° C., 2% CO₂, and 125 uE cool white LED lighting. For experiments relating to formate utilization, the above conditions and media were used, with varying CO₂ concentrations in a Percival Scientific growth chamber.

### Construct Assembly and Transformation

FDH variants utilized were mutated from the Pseudomonas sp. 101 FDH, specifically NAD+ utilizing variant (A198G) and NADP+ utilizing variant (A198G/D221Q/C255A/H379K/S380V). FDH transformation vectors were prepared by Twist Bioscience, cloning a ribosomal binding site (AGGAGGTTATAAAAA) and codon optimized FDH downstream of the ptxD selectable marker in our previously described chloroplast transformation vector. P. renovo transformation was carried out as described previously, with the exception that Critter Technology binding and precipitation buffers were used according to the manufacturers recommendations to bind DNA (plasmid prepared by Twist Bioscience) onto the gold microcarriers for biolistic transformation.

### Formate Quantitative Analysis

Formate quantification was carried out by high performance liquid chromatography using an Agilent 1100 series system. Six μL of filtered cell-free supernatant was used for injection into the Bio-Rad HPX-87H (300×7.8 mm) ion exchange column. Elution of the organic acid was carried out with 0.01 N sulfuric acid at a flow rate of 0.6 mL per min. The column temperature was maintained at 55° C. The retention peak time was recorded using Chemstation software followed by quantification using a standard curve generated for formate.

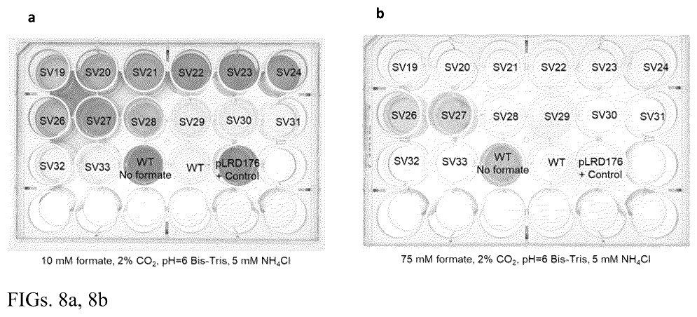

Additional formate dehydrogenase (FDH) enzymes were tested from diverse sources that also proved to be functional, as they reduced formate toxicity, and enabled growth in cultures with added sodium formate. Indeed, two of these FDHs SV26 and SV27 as labelled in FIGS. 8a and 8b were derived from Candida boidinii and were each able to grow on up to 75 mM sodium formate, whereas the other FDHs tested did not grow under these conditions. FIG. 8a depicts growth of FDHs in 10 mM formate, 2% CO₂ at pH=6 Bis-Tris and 5 mM NH₄Cl. FIG. 8b depicts growth of FDHs in 75 mM formate, 2% CO₂ at pH=6 Bis-Tris and 5 mM NH₄Cl.

### Sequences of FDHs

Pseudomonas sp. 101 WT (SV19) nucleotide sequence is SEQ ID NO: 1, the amino acid sequence of the expressed protein is SEQ ID NO: 2; Pseudomonas sp. 101 (D221Q/H223N) (SV20) nucleotide sequence is SEQ ID NO: 3, the amino acid sequence of the expressed protein is SEQ ID NO: 4, Mycobacterium vaccae FDH (C145S/D221Q/C225V) (SV21) nucleotide sequence is SEQ ID NO: 5, the amino acid sequence of the expressed protein is SEQ ID NO: 6, Paracoccus sp.12A-FDH-WT (SV22) nucleotide sequence is SEQ ID NO: 7, the amino acid sequence of the expressed protein is SEQ ID NO: 8, Ancylobacter aquaticus FDH (SV23) nucleotide sequence is SEQ ID NO: 9, the amino acid sequence of the expressed protein is SEQ ID NO: 10, Thiobacillus sp. FDH (SV24) nucleotide sequence is SEQ ID NO: 11, the amino acid sequence of the expressed protein is SEQ ID NO: 12, Candida boidinii FDH (C23S) (SV26) nucleotide sequence is SEQ ID NO: 13, the amino acid sequence of the expressed protein is SEQ ID NO: 14, Candida boidinii FDH (C23S/C262A) (SV27) nucleotide sequence is SEQ ID NO: 15, the amino acid sequence of the expressed protein is SEQ ID NO: 16, Saccharomyces cerevisiae FDH-WT (SV28) nucleotide sequence is SEQ ID NO: 17, the amino acid sequence of the expressed protein is SEQ ID NO: 18, Saccharomyces cerevisiae FDH (D196A/Y197R) (SV29) nucleotide sequence is SEQ ID NO: 19, the amino acid sequence of the expressed protein is SEQ ID NO: 20, Moraxella sp. C1-reco FDH (SV30) nucleotide sequence is SEQ ID NO: 21, the amino acid sequence of the expressed protein is SEQ ID NO: 22, Arabiodopsis thaliana FDH WT (SV31) nucleotide sequence is SEQ ID NO: 23, the amino acid sequence of the expressed protein is SEQ ID NO: 24, Gmax (Glycine max) FDH WT (SV32) nucleotide sequence is SEQ ID NO: 25, the amino acid sequence of the expressed protein is SEQ ID NO: 26, Gmax (Glycine max) FDH (F290D) (SV33) nucleotide sequence is SEQ ID NO: 27, the amino acid sequence of the expressed protein is SEQ ID NO: 28, Pseudomonas sp. 101 (A198G) (pLRD 176 (NAD FDH)) nucleotide sequence is SEQ ID NO: 29, the amino acid sequence of the expressed protein is SEQ ID NO: 30, Pseudomonas sp. 101 (A198G/D221Q/C255A/H379K/S380V) (pLRD 175 (NADP FDH)) nucleotide sequence is SEQ ID NO: 31, the amino acid sequence of the expressed protein is SEQ ID NO: 32.

Thus, as disclosed herein, formate is a potential next-generation renewable carbon source for phototroph cultivation. Also disclosed herein is the heterologous expression of formate dehydrogenase that decreases formate toxicity in P. renovo. Also disclosed herein is heterologous expression of formate dehydrogenase that enables formate utilization as a carbon source in P. renovo. Disclosed herein are methods and compositions of matter showing that formate supplementation enhances growth under ambient CO₂ cultivation in formate dehydrogenase expressing strains.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A non-naturally occurring phototrophic organism comprising a non-naturally occurring gene encoding for a formate dehydrogenase enzyme wherein the phototrophic organism can grow on formate as a sole carbon source.

2. The non-naturally occurring phototrophic organism of claim 1 wherein the non-naturally occurring gene has a nucleotide sequence that is greater than 70% identical to SEQ ID NO: 29, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17.

3. The non-naturally occurring phototrophic organism of claim 1 wherein the non-naturally occurring gene expresses a formate dehydrogenase enzyme that has an amino acid sequence that is greater than 70% identical to SEQ ID NO: 30, SEQ ID NO: 2, SEQ ID NO: 4, SEQ ID NO: 6, SEQ ID NO: 8, SEQ ID NO: 10, SEQ ID NO: 12, SEQ ID NO:
14, SEQ ID NO: 16, or SEQ ID NO: 18.

4. The non-naturally occurring phototrophic organism of claim 1 wherein the non-naturally occurring gene is incorporated into the plastidial genome of the phototrophic organism.

5. The non-naturally occurring phototrophic organism of claim 1 wherein the organism is Picochlorum renovo sp.

6. The non-naturally occurring phototrophic organism of claim 1 wherein the phototrophic organism exhibits increased growth in a medium comprising carbon dioxide and formate when compared to the corresponding naturally occurring phototrophic organism.

7. The non-naturally occurring phototrophic organism of claim 6 wherein the concentration of carbon dioxide in the medium is less than about 0.04 percent.

8. The non-naturally occurring phototrophic organism of claim 6 wherein the concentration of formate in the medium is greater than about 5 percent.

9. The non-naturally occurring phototrophic organism of claim 1 wherein the formate dehydrogenase enzyme uses NAD+ as a cofactor.

10. The non-naturally occurring phototrophic organism of claim 1 wherein the concentration of formate as a sole carbon source is greater than 10 mM.

11. The non-naturally occurring phototrophic organism of claim 1 wherein the concentration of formate as a sole carbon source is greater than 25 mM.

12. The non-naturally occurring phototrophic organism of claim 1 wherein the concentration of formate as a sole carbon source is greater than 70 mM.

13. A method for the growth of a non-naturally occurring phototrophic organism comprising using an electrolyzer to produce formate from carbon dioxide and then contacting the non-naturally occurring phototroph with the produced formate.

14. The method of claim 13 wherein the produced formate is at a concentration greater than 10 mM.

15. The method of claim 13 wherein the produced formate is at a concentration greater than 25 mM.

16. The method of claim 13 wherein the produced formate is at a concentration greater than 70 mM.

17. The method of claim 13 wherein the non-naturally occurring phototrophic organism is Picochlorum renovo sp.

18. The method of claim 13 wherein the non-naturally occurring phototrophic organism comprises a non-naturally occurring gene encoding for a formate dehydrogenase enzyme.

19. The method of claim 18 wherein the non-naturally occurring gene has a nucleotide sequence that is greater than 70% identical to SEQ ID NO: 29, SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 17.

20. The method of claim 18 wherein the non-naturally occurring gene expresses a formate dehydrogenase enzyme that has an amino acid sequence that is greater than 70% identical to SEQ ID NO: 30, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18.
